(19) United States
(12) Patent Application Publication
(10) Pub. No.: US 2020/0158679 A1
(43) Pub. Date: May 21, 2020

(54) ANALYTE SENSOR WITH EXTENDED LIFETIME

(71) Applicant: MEDTRONIC MINIMED, INC., Northridge, CA (US)

(72) Inventors: Santhisagar Vaddiraju, Plymouth, MN (US); Mark D. Breyen, Champlin, MN (US)

(21) Appl. No.: 16/194,076

(22) Filed: Nov. 16, 2018

(51) Int. Cl. G01N 27/327; G01N 33/543; A61B 5/145

(52) U.S. Cl. CPC G01N 27/3272; A61B 5/14532; G01N 33/5438; G01N 27/3273

(57) ABSTRACT

Disclosed herein are biosensors for sensing analyte concentration values, and methods of operating such sensors. The biosensor includes a probe. The probe includes a base substrate and a plurality of electrode sets overlying the base substrate. Each set of the plurality of electrode sets being individually operable for measuring a glucose concentration value when the probe is implanted in a patient. The probe also includes a biodegradable coating covering at least one electrode set of the plurality of electrode sets. The biodegradable coating does not cover at least one other electrode set of the plurality of the electrode sets.

### TECHNICAL FIELD

Embodiments of the subject matter described herein relate generally to sensors for measuring analyte concentrations. More particularly, embodiments of the subject matter relate to biosensors having an extended lifetime.

### BACKGROUND

Needle-implantable biosensors have shown to be useful for continuous analyte monitoring applications, such as glucose monitoring applications for use in diabetes management.

Some needle-implantable glucose biosensors operate by monitoring the amount of H₂O₂ which is produced from the catalyzed reaction of glucose by GOₓ to gluconic acid and H₂O₂ in the following reaction steps:
a) Glucose+GOₓ(FAD)→Glucorolactone+GOₓ(FADH₂)
b) GOₓ(FADH₂)+O₂→GOₓ(FAD)+H₂O₂

The product H₂O₂ is then electrochemically oxidized on the working electrode surface of a probe of the biosensor, thereby generating an electrical current response signal to be measured. The blood glucose concentration can be correlated to the current response signal obtained from the oxidation of the H₂O₂, or to the electrochemical reduction of O₂, via the reversible reaction:
c) H₂O₂ [symbol] 2H⁺+O₂+2e⁻

One prevalent type of biosensor is a biosensor that forms part of a transcutaneous system, and which measures subcutaneous interstitial glucose. Most biosensors of this type have FDA approval for a 3- to 7-day window of use. After this time, biosensors may become less accurate, due to, for example, biofouling of the sensor, which causes a decrease in the sensitivity of the biosensor.

Biofouling is a mechanism where sensor probes, at insertion, become exposed to blood, interstitial fluid, and blood borne- and interstitial fluid borne- constituents. When the sensor probe is exposed to these bodily-fluid borne- constituents, the sensor will become “fouled” with a layer of blood plasma proteins, adhered blood cells and glucose-consuming inflammatory cells, amongst other pollutants. This form of fouling is typically the first stage in the body's “foreign body response”. Subsequent stages of foreign body response may include capsulation of the sensor probe.

Biofouling typically decreases the diffusion of interstitial glucose to the sensor, thereby artificially decreasing the glucose concentration in the area surrounding the sensor probe. As such, the biosensor may detect an erroneous too-low amount of glucose, thereby leading to an incorrect glucose value being measured and shown to the user.

The typical time for a conventional sensor probe to become biofouled to the point where the sensitivity of the biosensor decreases to an unacceptable level is around 7 days. At this point, it is necessary for the user to change the biosensor, which may cause the user discomfort.

Accordingly, it is desirable to extend the lifespan of implanted biosensor probes.

Furthermore, other desirable features and characteristics will become apparent from the subsequent detailed description and the appended claims, taken in conjunction with the accompanying drawings and the foregoing technical field and background.

### BRIEF SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to a first exemplary embodiment, there is provided a sensor probe for a biosensor. The sensor probe includes a base substrate; and a plurality of electrode sets overlying the base substrate. Each set of the plurality of electrode set is individually operable for measuring an analyte concentration value when the probe is implanted in a patient. The probe also includes a biodegradable coating covering at least one electrode set of the plurality of electrode sets, wherein the biodegradable coating does not cover at least one other electrode set of the plurality of the electrode sets.

According to a second exemplary embodiment, there is provided a biosensor. The biosensor includes a sensor probe for a biosensor. The sensor probe includes a base substrate; and a plurality of electrode sets overlying the base substrate. Each set of the plurality of electrode set is individually operable for measuring an analyte concentration value when the probe is implanted in a patient. The probe also includes a biodegradable coating covering at least one electrode set of the plurality of electrode sets, wherein the biodegradable coating does not cover at least one other electrode set of the plurality of the electrode sets. The biosensor further includes an impulse generator, and wherein the biodegradable coating is operably connected to the impulse generator, the impulse generator operable to generate an impulse for initiating degradation of the biodegradable coating.

According to a third exemplary embodiment, there is provided a method of manufacturing a probe for a biosensor, the method includes the step of providing a base substrate. The method also includes the step of forming a plurality of electrode sets on the base substrate. The method also includes the step of covering at least one electrode set of the plurality of electrode sets with a biodegradable coating, whilst not covering at least one other electrode set of the plurality of electrode sets with the biodegradable coating.

According to a fourth exemplary embodiment, there is provided a method of operating a biosensor including a sensor probe for a biosensor. The sensor probe includes a base substrate; and a plurality of electrode sets overlying the base substrate. Each set of the plurality of electrode set is individually operable for measuring an analyte concentration value when the probe is implanted in a patient. The probe also includes a biodegradable coating covering at least one electrode set of the plurality of electrode sets, wherein the biodegradable coating does not cover at least one other electrode set of the plurality of the electrode sets. The method includes the step of obtaining, using the at least one first electrode set, analyte concentration measurements. The method also includes the step of assessing, using a processor, an extent of biofouling of the at least one first electrode set of the plurality of electrode sets. The method also includes the step of comparing, using a processor the determined extent of biofouling to a pre-determined threshold. The method also includes the step of generating, using an impulse generator, an impulse operable to initiate degradation of the biodegradable coating and, after degradation of the biodegradable coating. The method also includes the step of obtaining, using the at least one second electrode set, analyte concentration measurements.

### BRIEF DESCRIPTION OF THE DRAWINGS

A more complete understanding of the subject matter may be derived by referring to the detailed description and claims when considered in conjunction with the following figures, wherein like reference numbers refer to similar elements throughout the figures.

FIG. 1 is a schematic of a location of a glucose biosensor on a user in accordance with exemplary embodiments;

FIG. 2 is a cross-sectional schematic of a probe of a glucose biosensor in accordance with exemplary embodiments being embedded in a user's tissue;

FIG. 3 is a schematic of the sensor probe in accordance with exemplary embodiments;

FIG. 4 is another schematic of the sensor probe in accordance with exemplary embodiments;

FIG. 5 shows yet another schematic of the sensor probe in accordance with exemplary embodiments;

FIG. 6 shows a flowchart depicting a method in accordance with exemplary embodiments;

and

FIG. 7 shows a flowchart depicting a method in accordance with exemplary embodiments.

### DETAILED DESCRIPTION

The following detailed description is merely illustrative in nature and is not intended to limit the embodiments of the subject matter or the application and uses of such embodiments. As used herein, the word “exemplary” means “serving as an example, instance, or illustration.” Any implementation described herein as exemplary is not necessarily to be construed as preferred or advantageous over other implementations. Furthermore, there is no intention to be bound by any expressed or implied theory presented in the preceding technical field, background, brief summary or the following detailed description.

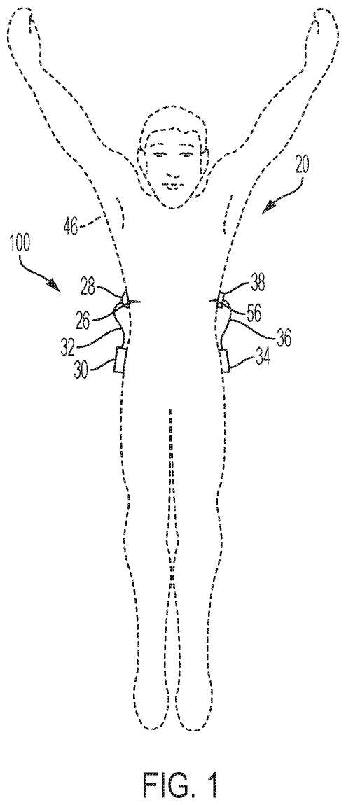

FIG. 1 shows a schematic of a continuous glucose monitoring system 100 being worn by a user 20. In an exemplary embodiment, the glucose monitoring system 100 includes a biosensor 28 having a probe 26, a characteristic monitor 30 and a sensor cable 32. In alternative embodiments, wireless data communication technology such as Wifi™ or Bluetooth®, or another method of wireless communication, can be employed instead of the physical sensor cable 32 in order to transmit data between the biosensor 28 and the characteristic monitor 30. In an exemplary embodiment, the glucose monitoring system 100 may be utilized together with an insulin administration device including an insulin infusion device 34 with an infusion channel 56, an infusion tube 36, and an infusion set 38. Moreover, the characteristic monitor 30 need not be utilized if the insulin infusion device 34 is configured to receive sensor data directly from the biosensor 28.

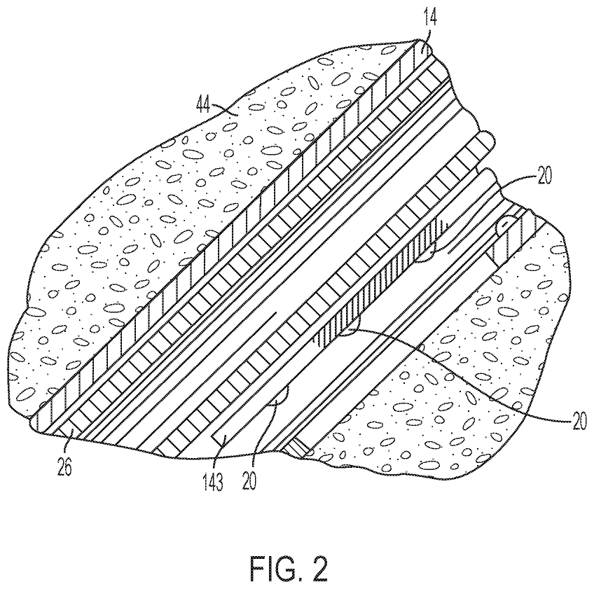

As shown in FIG. 2, the probe 26 of the biosensor is inserted through the user's skin into subcutaneous tissue 44 of a user using a needle 14. As can be seen in FIG. 2, the probe 26 includes an electrode set 143 that includes multiple individual electrodes 20, which are exposed to and in contact with interstitial fluid that is present throughout the user's subcutaneous tissue 44 when the probe 26 is first implanted into the user. The electrodes 20 include at least a working electrode and a counter electrode. The potential difference between the working electrode and the counter electrode caused by the electrochemical oxidation of H₂O₂ (or the electrochemical reduction of oxygen) on the working electrode can be used to determine a H₂O₂ (or oxygen) concentration value, which concentration value can then be used to determine a blood glucose concentration of the user. In exemplary embodiments, the electrodes 20 further include a reference electrode operable to maintain the voltage applied to the working electrode at a steady value. In particular, as is known, the voltage difference between the working electrode and the reference electrode may be measured and compared to a pre-determined value. When the measured voltage difference varies from the pre-determined value, it can be determined that the voltage applied to the working electrode has changed, and this voltage can be controlled back to the desired voltage value.

In an exemplary embodiment, the probe 26 further includes a catalyst which stimulates the reaction of blood glucose to gluconic acid and H₂O₂, which H₂O₂, may be used to determine glucose concentration in the manner described above. In some exemplary embodiments, insulin may be administered via the insulin infusion device 34 based on the determined blood glucose concentration.

In exemplary embodiments, the probe includes multiple electrode sets. A biodegradable coating (not shown in FIG. 2) is disposed over at least one of the electrode sets. At least one of the electrode sets is not covered by the biodegradable coating. The function of the biodegradable coating will be explained in more detail below.

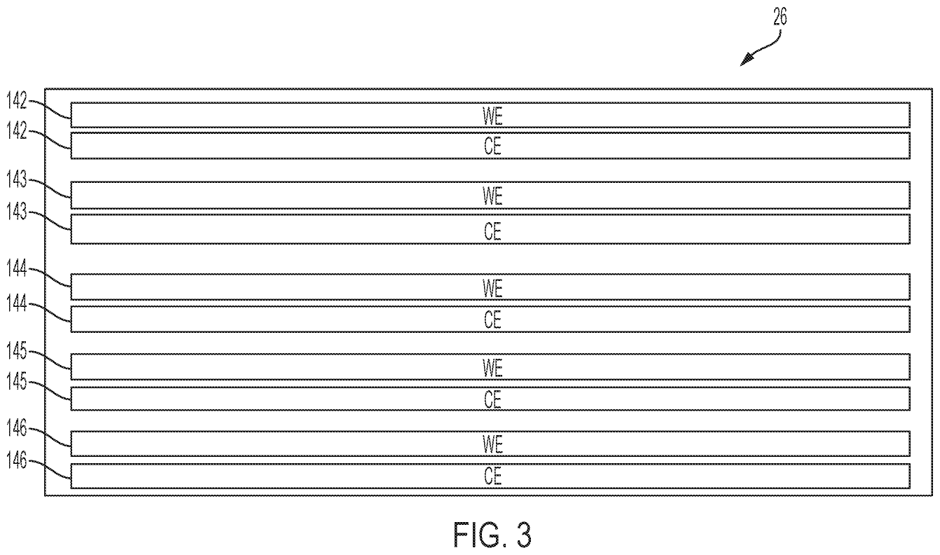

Turning to FIG. 3, this figure shows a top-down view of an exemplary sensor probe 26. As can be seen in FIG. 3, the sensor probe 26 includes a plurality of electrode sets 142, 143, 144, 145, and 146, with each electrode set including a working electrode (we) and a counter electrode (ce). As noted above, in exemplary embodiments a reference electrode is also included in each electrode set. Although five individual electrode sets are shown in FIG. 3, it will be appreciated that any number of electrode sets may be incorporated on the sensor probe 26, provided that there are at least two electrode sets on the sensor probe 26. Furthermore, although the electrode sets shown in FIG. 3 include only two electrodes (a working electrode and a counter electrode), it will be appreciated that more electrodes could be included in each electrode set. For example, in exemplary embodiments one or more of the electrode sets may additionally include a reference electrode.

Although the electrodes shown in each electrode set 142, 143, 144, 145, 146 of FIG. 3 are shown as strips of material, in exemplary embodiments the electrodes have different configurations, for example inter-digitated configurations. Inter-digitating the electrodes of each electrode set allows for an increase in the surface area of the electrodes between which mediator species can traverse, thereby increasing the strength of the signal measured when a voltage is applied to the working electrode and consequently improving the signal-to-noise ratio of each electrode set.

Although FIG. 3 shows each one of the plurality of electrode sets being disposed on only a first side one side of the sensor probe, in exemplary embodiments, the electrode sets are disposed on both a first side and a second side of the sensor probe 26. In particular, by disposing one or more of the electrode sets on a second side of the probe 26, opposite to the first side, multiple electrode sets may be accommodated on the probe 26 without substantially increasing the overall dimensions of the probe 26.

At least one of the electrode sets 142, 143, 144, 145, 146 is covered with a biodegradable coating (not shown in this figure). At least one of the electrode sets 142, 143, 144, 145, 146 is not covered with the biodegradable coating. Multiple electrode sets may be covered with different thicknesses of biodegradable coating, as shall be explained in more detail below.

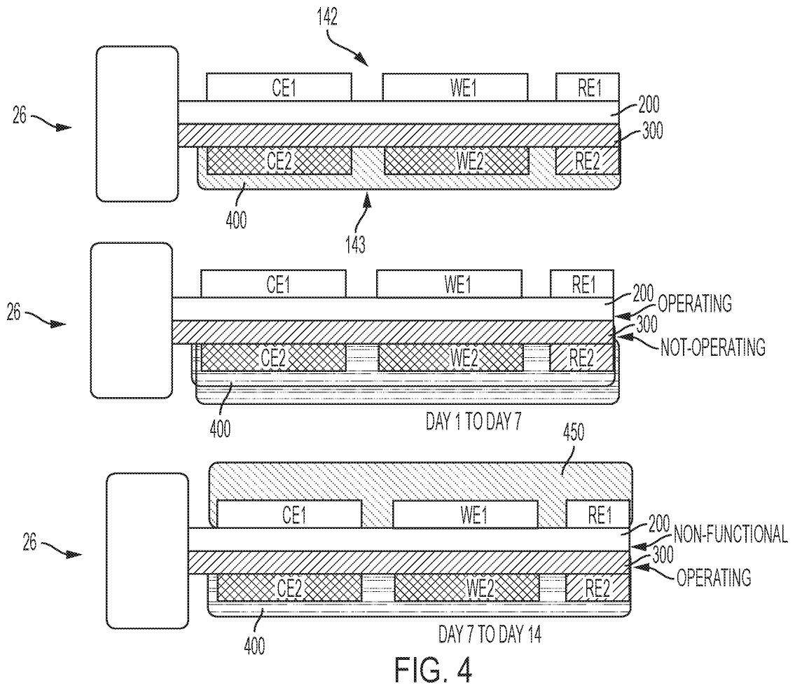

The function of the biodegradable coating will now be explained with reference to FIG. 4.

FIG. 4 shows three views of the same sensor probe 26. The sensor probe 26 has a first surface 200 and a second surface 300 opposite the first surface 200. A first electrode set 142 is disposed on the first surface 200 and a second electrode set 143 is disposed on the second surface 300. Each electrode set 142, 143 includes a working electrode WE1, WE2; a counter electrode CE1, CE2, and a reference electrode RE1, RE2. In exemplary embodiments, the first surface 200 and the second surface 300 of the probe 26 comprise two separate base substrates that are fastened to one another with a fastener, for example by gluing these two base substrates together.

The working electrode WE2, counter electrode CE2 and reference electrode RE2 of the electrode set 143 is covered with a biodegradable coating 400. The working electrode WE1, counter electrode CE1 and reference electrode RE1 of the other electrode set 142 of the electrode sets 142, 143 is not covered with a biodegradable coating 400, such that these electrodes are exposed to bodily fluids when implanted into tissue of a user.

In use, when the probe 26 is implanted into a user, glucose measurements are performed using the electrode set 142 which is not covered by the biodegradable coating 400. In other words, a voltage is applied to the working electrode WE1 of the electrode set 142 which is not covered with the biodegradable coating 400, and the current response is measured in a conventional manner.

The first electrode set 142 operates for a first time period, for example a number of days, before biofouling of the electrodes decreases the sensitivity of the biosensor to such an extent that the glucose concentrations measured by this electrode set 142 become inaccurate. For example, the first electrode set 142 may be operable for a period of seven days.

Whilst the first electrode set 142 is operating, the biodegradable coating 400 covering the second electrode set 143 gradually degrades, as represented in the middle drawing of FIG. 4. The middle drawing of FIG. 4 represents the biodegradable coating 400 degrading over time due to an interaction between the coating and bodily fluid of the patient. Whilst the biodegradable coating covers the second electrode set 143, the second electrode set is protected from biofouling, but cannot make any glucose concentration measurements since glucose and oxygen cannot diffuse through the biodegradable coating 400.

The thickness of the biodegradable coating 400 is selected such that the time taken for the coating to fully degrade corresponds to the time taken for the first electrode set 142 to become inoperative or inaccurate due to biofouling. As such, after this time period and full degradation of the coating 400, the second electrode set 143 is then exposed to the blood and interstitial fluid of the user and may then be used in the monitoring of glucose concentration levels of the user. This is represented in the bottom drawing of FIG. 4, which shows the first electrode set being rendered inoperable due to biofouling 450 and the second electrode set 143 is free from biofouling.

As such, the service lifespan of the sensor probe 26 of FIG. 4 is effectively doubled due to the inclusion of an additional electrode set 143 covered by a biodegradable coating. In exemplary embodiments, the sensor probe 26 includes more than two electrode sets, with varying biodegradable coating thicknesses on each electrode set. For example, the sensor probe 26 may contain three electrode sets, with a first electrode set having no biodegradable coating, a second electrode set having a biodegradable coating having a first thickness, and a third electrode set having a biodegradable coating having a second thickness of greater thickness than the first thickness. In use, the first electrode set will be used for measuring glucose concentrations after initial implantation of the probe into the user. When the electrodes of the first electrode set are biofouled to an extent where the sensitivity of the first electrode set has decreased, the biodegradable coating covering the second electrode set has degraded such that the second electrode set can then measure glucose concentrations of the user. When the second electrode set is biofouled to an extent where the sensitivity of the second electrode set has decreased, the biodegradable coating covering the third electrode set has degraded such that the second electrode set can measure glucose concentrations of the user.

In exemplary embodiments, the biodegradable coating is formed from a polymer made up of hydrophobic or hydrophilic blocks, or a combination of hydrophilic and hydrophobic blocks. For example, the hydrophobic, biodegradable blocks can be comprise of one or more of: Poly (lactic-co-glycolic acid), poly (lactic acid), Poly Glycolic acid, polyanhydrides, polyaspirins, etc. as well as combinations thereof. The hydrophilic blocks may be composed of one or more of poly vinyl alcohol, polyethylene oxide, polybetaines, polyacrylates, polyacrylamides, polyvinylacetates, etc. as well as combinations thereof.

In an exemplary embodiment, the molecular weight and thickness of the biodegradable coating can be varied such that the rate of degradation of the biodegradable coating matches the rate of biofouling of the sensors. The rate of biofouling of the sensors can be estimated by calculating the mean drop in sensor sensitivity over a population of multiple sensors over a fixed period of time and at a fixed glucose concentration. On the basis of this estimated rate of biofouling, the molecular weight and thickness of the biodegradable coating can be selected during formation of the biodegradable coating.

In the above-described embodiments, the biodegradable coating degrades naturally in vivo.

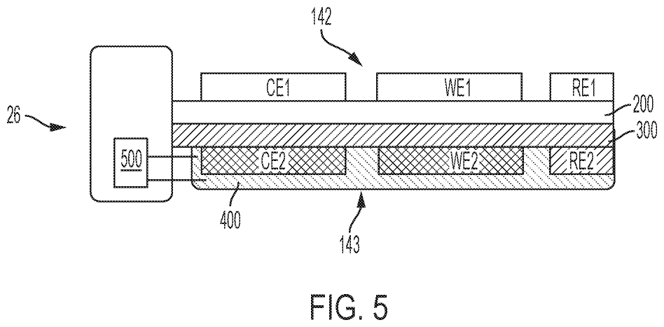

In alternative exemplary embodiments, a stimulus may be applied to the biodegradable coating in order to stimulate degradation of the coating at a specific time. In exemplary embodiments, as shown in FIG. 5, the biodegradable coating degrades responsive to an external impulse supplied by an impulse generator 500 operably connected to the biodegradable coating 400. In exemplary embodiments, the impulse generator 500 comprises a piezoelectric pulse generator operable to generate an electrical impulse. In alternative exemplary embodiments, the impulse generator 500 comprises a heat generator operable to generate an impulse comprising localized heat.

The biodegradable coating 400 degrades in response to the impulse generated by the impulse generator 500. In this manner, the degradation of the biodegradable coating 400 can be initiated when desired to allow for the coated electrode set 143 to begin glucose concentration measurements when an uncoated electrode set 142 is biofouled to such an extent that it no longer has the required degree of sensitivity. In an exemplary embodiment, a user may transmit a signal to the impulse generator 500 to generate an impulse to degrade the coating 400. Alternatively, the impulse generator 500 may automatically generate an impulse after a pre-determined time period (which time period may be determined in the same manner as described above) to degrade the coating 400.

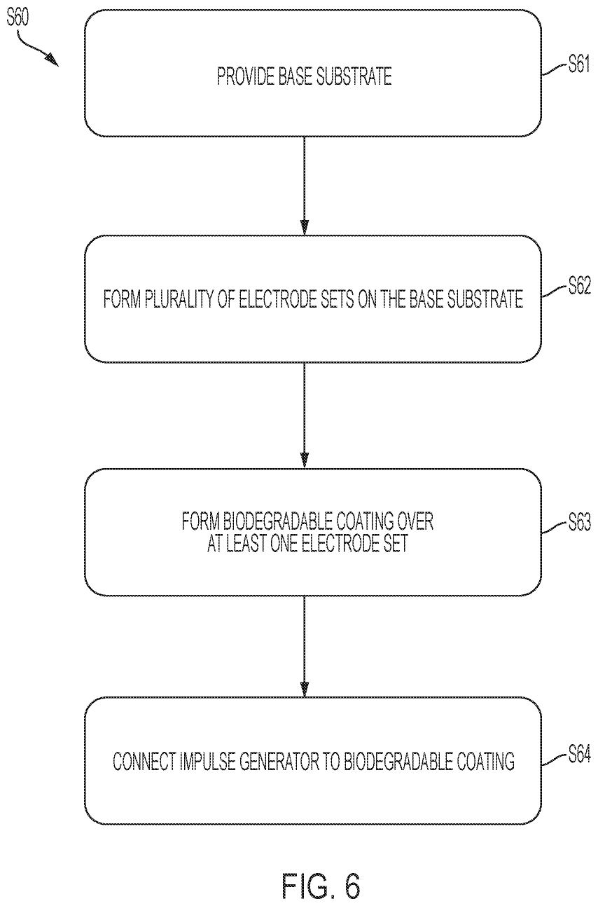

Turning to FIG. 6, a flowchart illustrating a method S60 of manufacturing a sensor probe in accordance with exemplary embodiments is shown. At step S61, at least one base substrate is provided. In exemplary embodiments, the at least one base substrate is formed from a bioinert material, for example polyester or polyethylene terephthalate (PET). The method then progresses to step S62.

At Step S62, a plurality of electrode sets are formed on the at least one base substrate. In embodiments, the plurality of electrode sets are formed by depositing a conductive material, for example platinum, onto the at least one base substrate and then forming electrode sets using this conductive material. In exemplary embodiments, the conductive material is deposited onto the at least one base substrate by sputtering or electroplating the conductive material onto the base substrate. In exemplary embodiments, the electrode sets are formed by etching or laser ablating the conductive material deposited onto the at least one base substrate. The method then progresses to step S63.

At Step S63, a biodegradable coating is formed over at least one electrode set of the plurality of electrode sets. In an exemplary embodiment, the biodegradeable coating is deposited over the electrode set by slot coating followed by patterning using photolithography. In an alternative exemplary embodiment, the biodegradable coating is deposited by slot coating over a pre-laid mask which can be later removed. In exemplary embodiments, the biodegradable coating is poly lactic glycolic acid. The method then may optionally progress to step S64.

In optional Step S64, an impulse generator is operably connected to the biodegradable coating such that an impulse generated by the impulse generator is operable to initiate degradation of the biodegradable coating.

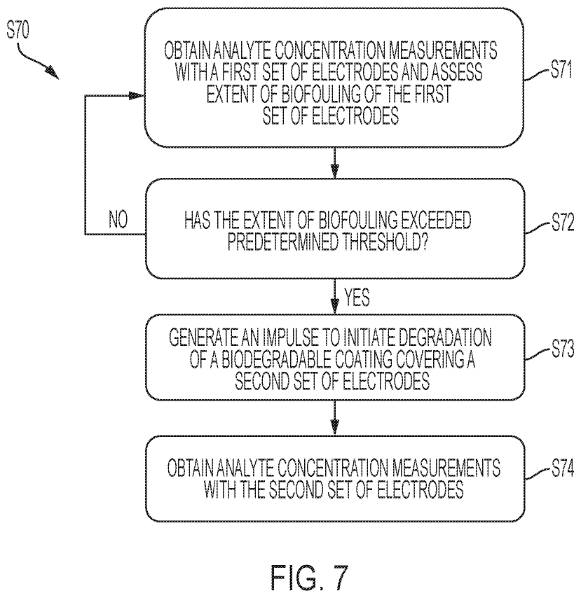

FIG. 7 shows a flowchart illustrating a method S70 of operating a sensor probe in accordance with exemplary embodiments.

At step S71, a first set of electrodes are used to obtain analyte concentration measurements.

The analyte of interest may be glucose. Concurrently, the extent of biofouling of the first set of electrodes is assessed. In exemplary embodiments, the extent of biofouling of the first set of electrodes is assessed by determining the time over which the electrodes have been exposed to blood and interstitial fluid. In alternative exemplary embodiments, the extent of biofouling of the first set of electrodes is assessed by looking at rate of change of the signal at a fixed glucose concentration and comparing it to a pre-determined look-up charts which tabulates the rate of signal change to changes in the electrode set or outer membrane. The method then progresses to step S72.

At step S72 a determination is made, using a processor, as to whether the extent of biofouling of the first electrode set has exceeded a pre-determined amount. In exemplary embodiments, this determination is made by comparing, using a processor, whether the first set of electrodes has been exposed to blood and interstitial fluid for a time longer than a pre-determined time. In exemplary embodiments, this determination is made by electronically comparing, using a processor, the rate of change of the signal to a pre-determined look-up chart. The processor may form part of an impulse generator or may be a separate component included in the biosensor. If, on the basis of this determination, it is determined that the extent of biofouling of the first electrode set is less than the pre-determined amount, the method reverts to Step S71. If, on the basis of this determination, it is determined that the extent of biofouling of the first electrode set is greater than the pre-determined amount, the method then progresses to step S73.

At step S73, an impulse is generated, using an impulse generator, and imparted to a biodegradable coating covering a second electrode set to initiate degradation of the biodegradable coating and thereby expose the second electrode set. After the biodegradable coating is degraded, the method progresses to step S74.

At step S74, the second set of electrodes are used to obtain analyte concentration measurements.

In addition, certain terminology may also be used in the following description for the purposes of reference only, and thus are not intended to be limiting. For example, terms such as “upper”, “lower”, “above”, and “below” refer to directions in the drawings to which reference is made. Terms such as “front”, “back”, “rear”, “side”, “outboard”, and “inboard” describe the orientation and/or location of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second”, and other such numerical terms referring to structures do not imply a sequence or order unless clearly indicated by the context.

For the sake of brevity, conventional techniques related to biosensor probe manufacturing may not be described in detail herein. Furthermore, the connecting lines shown in the various figures contained herein are intended to represent exemplary functional relationships and/or physical couplings between the various elements. It should be noted that many alternative or additional functional relationships or physical connections may be present in an embodiment of the subject matter.

While at least one exemplary embodiment has been presented in the foregoing detailed description, it should be appreciated that a vast number of variations exist. It should also be appreciated that the exemplary embodiment or embodiments described herein are not intended to limit the scope, applicability, or configuration of the claimed subject matter in any way. Rather, the foregoing detailed description will provide those skilled in the art with a convenient road map for implementing the described embodiment or embodiments. It should be understood that various changes can be made in the function and arrangement of elements without departing from the scope defined by the claims, which includes known equivalents and foreseeable equivalents at the time of filing this patent application.

1. A probe for a biosensor, the probe comprising:
a base substrate;
a plurality of electrode sets overlying the base substrate, each set of the plurality of electrode sets being individually operable for measuring an analyte concentration value; and
a biodegradable coating covering at least one electrode set of the plurality of electrode sets, wherein the biodegradable coating does not cover at least one other electrode set of the plurality of the electrode sets.

2. The probe of claim 1, wherein a thickness of the biodegradable coating is selected such that the biodegradable coating is operable to degrade over a pre-determined time period.

3. The probe of claim 2, wherein the pre-determined time period corresponds to a time period taken for the at least one other electrode set not covered by the biodegradable coating to become bio-fouled to an extent where the accuracy of measurements obtained by the at least one other electrode set not covered by the biodegradable coating is decreased by a pre-determined amount.

4. The probe of claim 1, wherein the base substrate comprises a first surface and a second surface opposite the first surface, wherein the at least one electrode set covered by the biodegradable coating is disposed on the first surface, and wherein the at least one other electrode set not covered by the biodegradable coating is disposed on the second surface.

5. The probe of claim 1, wherein the biodegradable coating is formed from a polymer made up of hydrophobic blocks or hydrophilic blocks, or a combination thereof.

6. A biosensor comprising:
a probe comprising:
a base substrate;
a plurality of electrode sets overlying the base substrate, each set of the plurality of electrode sets being individually operable for measuring an analyte concentration value;
a biodegradable coating covering at least one electrode set of the plurality of electrode sets, wherein the biodegradable coating does not cover at least one other electrode set of the plurality of the electrode sets; and
an impulse generator operably coupled to the biodegradable coating of the probe, the impulse generator operable to generate an impulse for initiating degradation of the biodegradable coating.

7. The biosensor of claim 6, wherein the impulse generator is operable to generate an electrical impulse.

8. The biosensor of claim 6, wherein the impulse generator is operable to generate a heat impulse.

9. The biosensor of claim 6, wherein the impulse generator is operable to generate the impulse in response to a determination that the at least one other electrode set not covered by the biodegradable coating has been biofouled to an extent where the accuracy of measurements obtained by the at least one other electrode set not covered by the biodegradable coating is decreased by a pre-determined amount.

10. The biosensor of claim 6, wherein the biodegradable coating is formed from a polymer made up of hydrophobic blocks or hydrophilic blocks, or a combination thereof.

11. The biosensor of claim 6, wherein the base substrate comprises a first surface and a second surface opposite the first surface, wherein the at least one electrode set covered by the biodegradable coating is disposed on the first surface, and wherein the at least one other electrode set not covered by the biodegradable coating is disposed on the second surface.

12. The biosensor of claim 6, wherein a thickness of the biodegradable coating is selected such that the biodegradable coating is operable to degrade over a pre-determined time period.

13. The biosensor of claim 6, wherein the pre-determined time period corresponds to a time period taken for the at least one other electrode set not covered by the biodegradable coating to become bio-fouled when exposed to bodily fluids to an extent where the accuracy of measurements obtained by the at least one other electrode set not covered by the biodegradable coating is decreased by a pre-determined amount.

14. A method of operating a biosensor, the biosensor having a probe comprising:
a base substrate;
a plurality of electrode sets overlying the base substrate, each one of the plurality of electrode sets being individually operable for measuring an analyte concentration value; and
a biodegradable coating covering at least one second electrode set of the plurality of the electrode sets, wherein the biodegradable coating does not cover at least one first electrode set of the plurality of the electrode sets, the method comprising:
obtaining, using the at least one first electrode set, analyte concentration measurements;
assessing, using a processor, an extent of biofouling of the at least one first electrode set of the plurality of electrode sets;
comparing, using a processor, the determined extent of biofouling to a pre-determined threshold;
generating, using an impulse generator, an impulse operable to initiate degradation of the biodegradable coating and, after degradation of the biodegradable coating;
obtaining, using the at least one second electrode set, analyte concentration measurements.

15. The method of claim 14, wherein the assessing step comprises comparing a time period over which the at least one first electrode set is exposed to bodily fluids to a pre-determined time period.

16. The method of claim 14, wherein the assessing step comprises determining, using a processor, a sensitivity level associated with the first electrode set and comparing the determined sensitivity level to a pre-determined sensitivity threshold.

17. The method of claim 14, wherein the generating step comprises generating an electrical impulse.

18. The method of claim 14, wherein the generating step comprises generating a heat impulse.

19. The method of claim 14, wherein the biodegradable coating is formed from a polymer made up of hydrophobic blocks or hydrophilic blocks, or a combination thereof.

20. The method of claim 14, wherein the base substrate comprises a first surface and a second surface opposite the first surface, wherein the at least one first electrode set is disposed on the first surface, and wherein the at least one second electrode set is disposed on the second surface.
